(19) United States
(12) Patent Application Publication
(10) Pub. No.: US 2013/0261381 A1
(43) Pub. Date: Oct. 3, 2013

(54) Devices and Methods for the Treatment of Obesity

(71) Applicant: Theodore M. BENDER, San Anselmo, CA (US)

(72) Inventor: Theodore M. BENDER, San Anselmo, CA (US)

(73) Assignee: VIBRYNT, INC., Redwood City, CA (US)

(21) Appl. No.: 13/437,644

(22) Filed: Apr. 2, 2012

(51) Int. Cl. A61F 2/00

(52) U.S. Cl. 600/37

(57) ABSTRACT

A device includes an expandable body having a planar configuration and a tongue for fastening the device. The body further includes at least one expandable region that contact a comparatively high surface area of the stomach as compared to its expanded volume. Methods for implanting the device and for treating a patient are provided.

### BACKGROUND OF THE INVENTION

Embodiments of the present invention relate generally to medical devices and methods and more particularly to minimally invasive devices, systems and methods for treating obesity.

Obesity has become a major health concern, both nationally and internationally. The National Center for Health Statistics (NCHS) estimates that over 120 million Americans are overweight, including about 56% of the adult population. Of these, about 52 million are considered obese, as measured by a body mass index (BMI) of 30% or greater. In Europe, an estimated 77 million people are obese, as measured by the same standard. This problem is not limited to western nations, as many developing countries are reported to have obesity rates over 75% of the adult population.

Co-morbidities that are associated with obesity include, but are not limited to type II Diabetes, high blood pressure, sleep apnea, stroke and arthritis, the symptoms of which often tend to be lessened or alleviated upon loss of weight by a person so affected.

In the U.S., options for treatment of obesity are currently quite limited. Current treatment methodologies typically rely upon surgically introducing a “malabsorptive” environment in the gastro-intestinal tract, a restrictive environment, or a combination of these. One available treatment method is gastric bypass surgery and another is referred to as gastric banding (one of these techniques if referred to as the LAPBAND™ procedure). These procedures are limited to only those patients with a BMI over 40 (or over 35, with co-morbidities present).

Gastric bypass procedures incur a great deal of morbidity and create a malabsorptive state in the patient by bypassing a large portion of the intestines. Serious side effects, such as liver failure have been associated with this procedure, as well as chronic diarrhea. Another surgical procedure that has a high degree of morbidity associated with it is known as the “Gastric Bypass Roux-en-Y” procedure. This procedure reduces the capacity of the stomach by creating a smaller stomach pouch. The small space holds only about one ounce of fluid. A tiny stomach outlet is also surgically created to slow the speed at which food leaves the stomach. Staples are used to create a small (15 to 20 cc) stomach pouch, with the rest of the stomach being stapled completely shut and divided from the stomach pouch. The small intestine is divided just beyond the duodenum, brought up, and connected to the newly formed stomach pouch. In addition to the considerable morbidity associated with this procedure, other disadvantages include “dumping syndrome”, where stomach contents are literally “dumped” rapidly into the small intestine which may lead to nausea, weakness, sweating, faintness, and diarrhea; hernias resulting from the surgery; gallstones; leakage of the connection between the pouch and the intestine; stretching of the pouch that was formed; nutritional deficiencies; and possible dehiscence of the staples.

The LAPBAND™ is a band that, when placed, encircles the fundus-cardia junction and is inflatable to constrict the same. It does not reduce the volume of the stomach, but rather restricts passage of food into the stomach, the theory being that the patient will feel satiety with a much less volume of food than previously. Although the LAPBAND™ procedure is less invasive than a gastric bypass procedure, it also typically achieves less weight loss. Further, it is not a simple procedure and requires a substantial amount of training by a surgeon to become proficient in performing the procedure. Also, a substantial amount of dissecting and suturing is required because the pathway by which the band is introduced is not an existing pathway, and must be established by dissection. Great care is required to avoid blood vessels and nerves that may be in the intended pathway to be created by the dissection. After placing the band around the fundus-cardia junction, the ends of the band must be connected together and then it must be cinched down into place. Additionally, complications such as erosion at the fundus-cardia junction, slippage of the band from its intended location, nausea/vomiting, gastroesophageal reflux, dysphagia and lack of effectiveness in causing weight loss have been reported.

Intra-gastric balloons have also been placed, in an attempt to fill a portion of the volume in the stomach, with the theory being that it will then require less food than previously, to give the patient a sensation of fullness or satiety. This procedure involves delivery of a balloon (typically, trans-orally) to the interior of the stomach and inflation of the balloon to take up a portion of the volume inside the stomach. However, intra-gastric balloons may also lead to complications such as obstruction, vomiting and/or mucosal erosion of the inner lining of the stomach. The balloon can break down over extended exposure to the stomach's acids, and in some cases, after breaking down, the balloon translated through the intestines and caused a bowel obstruction.

Gastrointestinal sleeves have been implanted to line the stomach and/or a portion of the small intestines to reduce the absorptive capabilities of the small intestine and/or to reduce the volume in the stomach, by reducing the available volume to the tubular structure of the graft running therethrough. Although weight loss may be effective while these types of devices are properly functioning, there are complications with anchoring the device within the stomach/GI tract, as the stomach and GI tract function to break down things that enter into them and to move/transport them through. Accordingly, the integrity of the anchoring of the device, as well as the device itself may be compromised over time by the acids and actions of the stomach and GI tract.

A sleeve gastrectomy is an operation in which the left side of the stomach is surgically removed. This results in a much reduced stomach which is substantially tubular and may take on the shape of a banana. This procedure is associated with a high degree of morbidity, as a large portion of the stomach is surgically removed. Additionally, there are risks of complications such as dehiscence of the staple line where the staples are installed to close the surgical incisions where the portion of the stomach was removed. Further, the procedure is not reversible.

In the laparoscopic duodenal switch, the size of the stomach is reduced in similar manner to that performed in a sleeve gastrectomy. Additionally, approximately half of the small intestine is bypassed and the stomach is reconnected to the shortened small intestine. This procedure suffers from the same complications as the sleeve gastrectomy, and even greater morbidity is associated with this procedure due to the additional intestinal bypass that needs to be performed. Still further, complications associated with malabsorption may also present themselves.

An inflatable gastric device is disclosed in U.S. Pat. No. 4,246,893, in which a balloon is inserted anteriorly of the stomach and posteriorly of the left lobe of the liver. The balloon is then inflated to compress the stomach so that it fills with less food that would ordinarily be possible. Not only does this device compress the stomach, but it also compresses the liver, as seen in FIG. 5 of the patent, which may cause complications with the liver function. Additionally, the balloon is simply placed into this location, and there is no assurance that it will not migrate and lose its effectiveness in compressing the stomach to the degree intended. Still further, the balloon is of a simple spherical design, and, as such, extends pressure outwardly in all directions, 360 degrees in all planes. Accordingly, the liver is compressed just as much as the stomach is. Also, the compression forces against the stomach are not ideal, as the spherical balloon conformation does not match the conformation of the expanding stomach. The stomach is not spherical when expanded, or concave with a constant radius of curvature, but expands into a designated space that allows the fundus to expand preferentially more than other parts of the stomach.

Brazzini et al. in WO2005/18417 discloses at least two or more expandable devices used to treat obesity, in which the devices are inserted through the abdominal wall and anchored subcutaneously or to the stomach wall to exert pressure against the external surface of the stomach wall.

U.S. Patent Publication No. 2005/0261712 to Balbierz et al. describes capturing a device against the outer surface of the stomach wall to form a restriction that appears to function similarly to the restriction imposed by the LAPBAND™. The anchoring of the devices disclosed relies upon placement of features against the internal wall of the stomach to form an interlock with the device which is placed against the external wall of the stomach.

U.S. Patent Publication No. 2005/0267533 to Gertner discloses devices for treatment of obesity that use one or more anchoring mechanisms that are passed through the wall of the stomach to establish an anchor. The stomach is reduced in size by passing the devices through the stomach wall on opposite sides of the stomach and compressing the walls together to eliminate a portion of the interior space within the stomach. Gertner also discloses an embodiment in which an extra-gastric balloon is placed anteriorly of the stomach and attached to the abdominal wall using one of the anchoring mechanisms described.

U.S. Pat. No. 6,981,978 to Gannoe discloses devices for reducing the internal cavity of the stomach to a much smaller volume, which may be used to carry out a bypass procedure. Stapling is employed to isolate the smaller volume in the stomach, and thus the same potential disadvantages are present as with other stapling procedures described herein.

U.S. Pat. No. 6,186,149 to Pacella et al. describes an occluder device that can be used as a dietary control device (see FIG. 8C). The occluder device is placed against the wall of the stomach and inflated to press inwardly on the stomach wall. A frame is wrapped around the stomach wall and is inflated to press against the stomach wall. However, there is no disclosure of how the frame might be adjusted to maintain a position relative to the stomach wall as the size of the stomach varies.

Gastric reduction techniques have been attempted, such as by inserting instruments trans-orally and reducing the volume of the stomach by stapling portions of it together. However, this technique is prone to failure due to the staples pulling through the tissues (i.e., dehiscence) that they are meant to bind.

Techniques referred to as gastric pacing endeavor to use electrical stimulation to simulate the normal feedback mechanisms of a patient that signal the brain that the patient is full, or satiated. While these techniques are less invasive than some of the other existing treatments, statistics to date have shown that the amount of weight lost by using such techniques is less than satisfactory.

Currently marketed drugs for weight loss, such as XENICAL®, MERIDIA® and Phen fen have largely failed, due to unacceptable side effects and complications, and sometimes to an ineffective amount of weight loss. Other drugs that are on the horizon include ACCOMPLIA® and SYMLIN®, but these are, as yet, unproven.

Methods that places devices such as bands, balloons, or wraps on the extra-gastric surface of the stomach carry the risk of creating erosions on the stomach surface. Such erosions can lead to migration of the device, even through to the stomach. Erosions carry the risk of spillage of stomach content into the abdominal cavity, which can be a life-threatening event. Erosions can be caused by sharp edges or focused pressure on the stomach wall. Constriction of the stomach applies force to the stomach wall and devices that do so can create erosions.

The risk and invasiveness factors of currently available surgeries are often too great for a patient to accept to undergo surgical treatment for his/her obesity. Accordingly, there is a need for less invasive, yet effective surgical treatment procedures for morbidly obese patients (patients having a BMI of 35 or greater). Also, since the current surgical procedures are currently indicated only for those patients having a BMI of 40 or greater, or 35 or greater when co-morbidities are present, it would be desirable to provide a surgical procedure that would be available for slightly less obese patients, e.g., patients having a BMI of 30 to 35 who are not indicated for the currently available surgical procedures. It would further be desirable to provide a surgical procedure that would be indicated for obese patients having a BMI in the range of 30-35, as well as for more obese patients.

There is a need for devices and methods for treating obesity with minimally invasive devices that substantially preserve the physiological integrity of the blood vessels around the stomach while providing an adjustable, constrictive force on the stomach.

### BRIEF SUMMARY OF THE INVENTION

Certain embodiments of the present invention are related to a device for the treatment of obesity. In certain embodiments, device includes a planar body having a configuration suitable for being positioned about the exterior of the stomach of a patient. The body includes an expandable region, a tongue region and a fastening region configured to fasten the tongue region. In certain embodiments, the body includes a folding region positioned between the expandable region and the tongue region and a reinforcing region. In certain embodiments, the body maintains a substantially planar configuration following expansion of the expandable region. In certain embodiments, the body includes at least one expandable region having surface area to internal volume ratio of greater than about 1.1:1 when expanded. In certain embodiments, the body includes a lateral retainer.

Certain embodiments of the present invention are related to a method for the treatment of obesity. The method includes accessing an extra-gastric abdominal space of a patient and introducing a device into the extra-gastric abdominal space. The device includes an expandable region having a substantially planar configuration suitable for being positioned about the exterior of the stomach of the patient and a tongue region. The method includes positioning the expandable region to be in contact with at least a portion of a first surface of the stomach and positioning the tongue region to be in contact with at least a portion of a second surface of the stomach, wherein one of the first surface and the second surface is an anterior surface of the stomach and the other is a posterior surface of the stomach. The method includes fastening the tongue region to the expanding region and expanding the expandable region.

### BRIEF DESCRIPTION OF SEVERAL VIEWS OF THE DRAWINGS

FIG. 1 illustrates the stomach, several of the blood vessels, and the connective tissue present near the stomach.

FIG. 2 illustrates a perspective view of device 100 according to certain embodiments of the invention.

FIG. 2A illustrates a cross-sectional view of planar body 110 according to certain embodiments of the invention.

FIG. 3 illustrates a perspective view of the exterior side of device 100 according to certain embodiments of the invention.

FIG. 4 illustrates a perspective view of a method of use of device 100 according to certain embodiments of the invention.

FIG. 5 illustrates a perspective view of device 100 being advanced into position around a stomach according to embodiments of the invention.

FIG. 6 illustrates a perspective view of device 100 being positioned to constrain or constrict a stomach according to embodiments of the invention.

FIG. 7 illustrates a perspective view of device 100 being fastened into position to apply constraining or constricting forces to a stomach according to embodiments of the invention.

FIG. 8 illustrates a perspective view of device 100 being expanded to apply constraining or constricting forces to a stomach according to embodiments of the invention.

FIG. 9 illustrates a perspective view of device 100 having lateral retainer according to embodiments of the invention.

FIG. 10 illustrates a perspective view of device 100 being fastened into position to apply constraining or constricting forces to a stomach according to embodiments of the invention.

### DETAILED DESCRIPTION OF THE INVENTION

Embodiments of the present invention are useful in the surgical treatment of obesity. The description, figures, and examples herein relate to devices and methods for the containment or constriction of the stomach.

Before the present devices and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

Short summaries of certain terms are presented in the description of the invention. Each term is further explained and exemplified throughout the description, figures, and examples. Any interpretation of the terms in this description should take into account the full description, figures, and examples presented herein.

The singular terms “a,” “an,” and “the” include plural referents unless the context clearly dictates otherwise. Thus, for example, reference to an object can include multiple objects unless the context clearly dictates otherwise. Similarly, references to multiple objects can include a single object unless the context clearly dictates otherwise.

The terms “substantially,” “substantial,” and the like refer to a considerable degree or extent. When used in conjunction with an event or circumstance, the terms can refer to instances in which the event or circumstance occurs precisely as well as instances in which the event or circumstance occurs to a close approximation, such as accounting for typical tolerance levels or variability of the embodiments described herein.

The terms “planar,” “plane,” and the like refer to an object or region of an object whose shape in a Cartesian coordinate system has a z-axis dimension substantially less than its x-axis and y-axis dimensions while its x-axis and y-axis dimensions are relatively similar. As a non-limiting example, a plate-like shape is considered planar, and the plate surface can be rectangular, circular, triangular or any other shape and still be considered planar. It is understood that an object can be planar and (i) be non-rigid and capable of flexion in three dimensions; (ii) have variation in its z-axis dimension; (iii) be capable of conforming to the shape of the anterior or posterior surface of a full stomach; (iv) have expanded and unexpanded configurations, both of which may be considered planar; or (v) combinations thereof.

The term “atraumatic” and the like refers to the characteristics of the features of a device that reduce the likelihood that such feature will cause trauma to tissue. Such characteristics include, but are no limited to, softness, flexibility, and bluntness. It is understood that atraumatic features are not limited to those than never cause injury or cause no injury.

The terms “fasten,” “fastening,” “fastener,” and the like refer to one part being coupled to another part in a substantially fixed manner. The two parts may be attached directly or through one or more intermediate parts provided that the intermediate parts preserve the substantially fixed manner of the coupling.

The terms “expand,” “expanded,” “expandable,” “expanding,” “expansion,” and the like refer to an object or region of an object becoming larger in size or volume. In certain cases herein, such expansion is accomplished by at least partially filling an internal volume of the object or region. However, it is understood that these terms include cases where an internal volume of the object or region is filled but the object or region does not stretch or grow. In such cases where filling the object does not cause the object or region to stretch or grow, the act of at least partially filling the internal volume of the object or region is the act of expansion. Conversely, the term “unexpanded” and the like refers to both the condition of an object or a region prior to it becoming larger in size or volume and to the condition of an object or region prior to its internal volume being at least partially filled.

A “compliant” material refers to a material that is stretchable or expandable. This expansibility allows the material to increase in dimension substantially more than a noncompliant or semi-compliant material, prior to failure. For example, when formed as an expandable body, a compliant material comprises an expansibility property of being able to increase its radius, beyond its formed radius, under pressure applied into the expandable body, by 100 percent or more, without rupturing.

A “noncompliant” material refers to a material that, when formed as an expandable body, can increase its radius beyond its formed radius, under pressure applied into the expandable body, only up to about 10 percent or less prior to rupturing.

A “semi-compliant” material refers to a material that, when formed as an expandable body, can increase its radius beyond its formed radius, under pressure applied into the expandable body, by an amount between about 10 percent and about 100 percent, prior to rupturing.

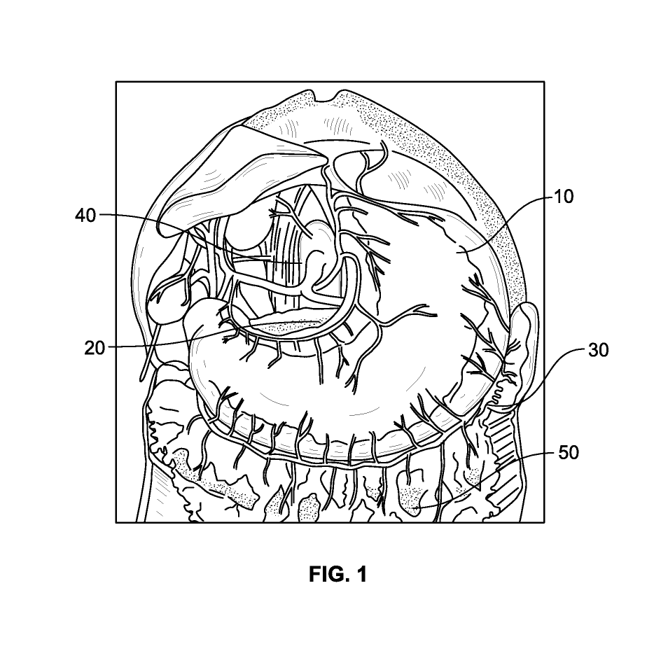

FIG. 1 illustrates stomach 10, several of the blood vessels, and the connective tissue present near stomach 10. Some of these blood vessels supply blood to the stomach itself and other blood vessels supply blood to internal organs such as the spleen or the liver. For example, located along or nearby the inner curvature 20 (or lesser curvature) of the stomach are the left gastric artery, the celiac trunk, the common hepatic artery, the aorta, and the right gastric artery. Other smaller branch arteries emanate from some of these arteries, making the inner curvature 20 an area rich with blood vessels important for the function of abdominal organs. Similarly, located along or nearby the outer curvature 30 (or greater curvature) of the stomach are the right and left gastroepoploic arteries, sections of the splenic artery, and a series of short gastric arteries. As compared to inner curvature 20, outer curvature 30 has a lower density of blood vessels, both those that supply the stomach 10 and those that supply other organs or muscles. In abdominal surgeries that involve the stomach, the area around the outer curvature tends to tolerate surgical dissection more readily than the area around the inner curvature in part because of the lower density of blood vessels in the area around the outer curvature.

Referring still to FIG. 1, connective tissue is present in the areas of both inner curvature 20 and outer curvature 30. Lesser omentum 40 is present in the area around inner curvature 20 and greater omentum 50 is present in the area around outer curvature 30. As used herein, “omentum” refers to lesser omentum 40, greater omentum 50, or both as the context dictates.

One of the novel aspects of the present invention is an appreciation of the importance of substantially maintaining the integrity of the blood vessels and connective tissue on or near both the inner curvature and the outer curvature of the stomach while still providing an effective treatment for obesity. Unlike devices and methods of the prior art that often depict and treat the stomach as a freestanding organ with little to no regard for the important blood vessels attached to or nearby it, certain embodiments of the present invention are configured to conform with the actual anatomy of the stomach and its milieu. Unlike devices and methods of the prior art, certain embodiments of the present invention are configured to facilitate the preservation of many of these blood vessels and much of the connective tissue both during the initial implantation of the device and during the lifetime of the device.

An advantage of certain devices and methods of the present invention as described in detail herein is that stomach 10 can be constricted or constrained without the blood supply to the stomach being cut off due to compression or dissection of blood vessels. As compared to some prior art instances of stomach constriction or constraint, the design, configuration, and execution of devices and methods of certain embodiments of the invention substantially avoid compressing or dissecting major blood vessels by allowing parts of the device to be placed away from the blood vessels yet also to apply and to maintain the necessary constrictive and/or constraining conditions. The advantage of preserving the blood vessels to the stomach is that it reduces the likelihood of the treatment cutting off blood flow (to and from the stomach) to a section of the stomach, which can lead to ischemia, which can cause leaks in the stomach, which can lead to an emergency surgery where much of the stomach may need to be removed. An advantage to preserving the connective tissues to the stomach is that the connective tissues help maintain the position of the stomach, and reduce the likelihood of the stomach twisting or curling up on itself, as can happen if the connective tissues are removed. Thus preservation of the vessels and connective tissue helps to avoid serious medical complications that are associated with bariatric surgeries that do not preserve these tissue connections.

Similarly, an advantage of certain devices and methods of the present invention as described in detail herein is that stomach 10 can be constricted or constrained without complete dissection of the connective tissue near stomach 10. Advantageously, the presence of substantially intact connective tissue can help devices of certain embodiments of the invention maintain the necessary constrictive and/or constraining conditions.

Another advantage of certain devices and methods of the present invention as described in detail herein is the relatively wide distribution of constrictive or constraining forces about stomach 10. The relatively high level of surface contact of certain embodiments of the device with the stomach allows for constrictive or constraining forces to be spread over a relatively larger surface area of the stomach as compared to certain prior art devices. Spreading a force over a relatively larger surface area decreases the amount of force that any given point within that surface area experiences. Without being bound by any particular theory or mode of action, when this concept is applied to the stomach through devices and methods of the invention the constricted or constrained stomach is less likely to experience erosions.

Yet another advantage of certain devices and methods of the present invention as described in detail herein is the use of relatively smooth contact points where the device interacts with the stomach. Certain devices of the prior art have relatively sharp or abrupt edges or device features that can act as focal points for forces on the stomach. Such focal points can lead to erosions. The various parts of certain embodiments of the invention are designed and configured to present rounded or “soft” edges and features to the stomach and the blood vessels in the area of the stomach so as to reduce the incidence of erosions and the compression or dissection of blood vessels and connective tissue.

Another advantage of certain embodiments of the present invention is that the device can conform to the stomach in such a way that the device moves with the stomach. When the device moves with the stomach it can substantially maintain its constrictive or constraining function even when the stomach moves. The conforming and adjustable nature of certain embodiments of the invention allows the device to move with the stomach.

Another advantage of certain embodiments of the present invention is that the device fastens to itself about the stomach in such a way that the device moves with the stomach. The device can be fitted such that the device remains in contact with the stomach whether the stomach is empty, full, or some state in between. Maintaining contact with the stomach is preferable to prevent the stomach from migrating away from the device and to prevent or decrease the incidence of erosions or other irritation of the stomach wall as compared to prior art devices. Further, in embodiments of the invention in which the device is folded or wrapped across or near an inferior region of the stomach, the device can help prevent migration of the stomach in the inferior direction.

Optionally, the device may also be anchored to the stomach at one or more locations around the stomach using conventional anchoring techniques. Optionally, the device may also be anchored to the abdominal wall at one or more locations using conventional anchoring techniques. Anchoring the device to the abdominal wall can also help prevent migration of the stomach, including in the inferior direction. By anchoring the device, which is fitted close to the stomach, the stomach is thereby also fixed in place as compared to an unanchored stomach.

According to certain embodiments of the invention, devices may be dimensioned and configured to be inserted using minimally invasive surgical techniques known the art. Devices may be inserted laproscopically, transorally, or through other means or procedures for accessing the extra-gastric abdominal space.

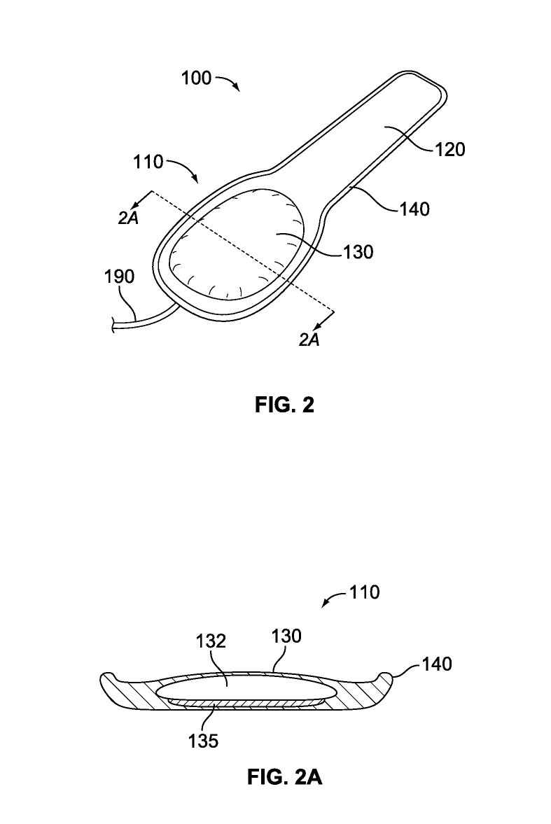

FIG. 2 illustrates a perspective view of device 100 according to certain embodiments of the invention. Device 100 includes planar body 110 which includes tongue region 120 and expandable region 130. Planar body 110 is generally flat when expandable region 130 is unexpanded. When expandable region 120 is expanded, planar body 110 is still generally planar due to the relatively low profile of expandable region 120 even when expandable region 130 is expanded. At or near the periphery of device planar body 110 is atraumatic region 140.

Referring still to FIG. 2, according to certain embodiments of the invention device 100 includes connector 190. Connector 190 is in fluid communication with the internal volume of expandable body 100 and facilitates the expansion and adjustment of expandable region 130. Connector 190 has a length to allow a proximal end portion thereof to extend out of the body of the patient when device 100 is implanted in the desired location and orientation in the abdominal space of a patient and expanded. Connector 190 may run along the surface of expandable region 130 and may be free of the surface of expandable region 130 except for where it inserts through the wall of expandable region 130. According to certain alternate embodiments of the invention, connector 190 may be fixed at one or more locations along its length that is adjacent to expandable region 130, or the entire adjacent length may be fixed to the surface of expandable region 130, such as by adhesive, vulcanization, welding, taping, or other mechanical fixation.

Expandable region 130 can be expanded with gas or liquid or both. Examples of gases or liquids that can be used to expand expandable region 130 include, but are not limited to: carbon dioxide, helium, isotonic dextrose solution, isotonic saline solution, and air. Connector 190 allows for expandable region 130 to be adjusted after implantation.

According to certain embodiments of the invention, expandable region 130 is formed of silicone. Alternatively, expandable region 130 may be formed by co-extrusion, e.g., co-extruding EVOH (ethylene-vinyl alcohol copolymer) and polyurethane. As another alternative embodiment, expandable region 130 may be formed of a blend of silicone and polyurethane. As a further alternative, expandable region 130 can be formed from or include one or more semi-compliant or non-compliant materials. Examples of useable semi-compliant materials include, but are not limited to: nylon, polyethylene, polyester, polyamide and polyurethane. Polyurethane, nylon, polyethylene and polyester can be compliant or semi-compliant materials, depending upon the specific formulation and hardness or durometer of the material as produced. Examples of noncompliant materials that can be used in the construction of expandable bodies described herein include, but are not limited to: polyethylene terepthalate (PET), polypropylene, poly ether-ether ketone, polysulfone and urethane.

Expandable region 130 has a radial thickness in its unexpanded condition and a radial thickness it its expanded condition. In some embodiments, the radial thickness of expandable region 130 in its unexpanded condition ranges from about 0.2 cm (0.08 inches) to about 1.5 cm (0.6 inches). Preferably, the radial thickness of expandable region 130 in its unexpanded condition ranges from about 0.4 cm (0.16 inches) to about 1 cm (0.4 inches). In some embodiments, the radial thickness of expandable region 130 in its expanded condition ranges from about 0.6 cm (0.2 inches) to about 6 cm (2.4 inches). Preferably, the radial thickness of expandable region 130 in its expanded condition ranges from about 1 cm (0.4 inches) to about 2.5 cm (1 inch). In some embodiments, the wall of expandable region 130 has a thickness that ranges from about 0.1 cm (0.04 inches) to about 0.8 cm (0.3 inches). Preferably, the wall of expandable region 130 has a thickness that ranges from about 0.2 cm (0.08 inches) to about 0.5 cm (0.2 inches).

FIG. 2A illustrates a cross-sectional view of planar body 110 according to certain embodiments. FIG. 2A illustrates a cross-section of expandable region 130, which includes expandable internal volume 132 and abuts reinforcing region 135. FIG. 2A depicts reinforcing region 135 near to the exterior surface of device 100 where exterior refers to the side of device 100 not in contact with the surface of the stomach. It is understood that reinforcing region 135 may directly abut expandable internal volume 132, may be spaced apart from expandable internal volume 132, or may be at or near the exterior surface of planar body 110. Further, planar body 110 may include one or more reinforcing regions 135.

The reinforcing region of certain embodiments of the invention can resist expansion under conditions that cause some or all of the rest of the expandable region to expand. According to certain embodiments, the reinforcing region may alternately or additionally influence the direction of expansion of the expandable region under conditions that cause the rest of the expandable region to expand. Generally speaking, the expandable region will expand preferentially away from the reinforcing region. In certain embodiments, such reinforcing regions are less compliant than the region of the expandable region that they reinforce.

The reinforcing regions can be made by any of the following methods or similar methods. In some embodiments, the reinforcing regions are composed of multiple layers of material. The layers of material can be added to the exterior surface of the planar body wall or to the interior surface of expandable internal volume 132. In some embodiments, expandable region 130 is turned inside out to facilitate the addition of multilayer reinforcing regions and then returned to its proper configuration. In other embodiments, multilayer reinforcing regions are added to the interior surface of expandable internal volume 132 through openings in expandable region 130 and such openings are subsequently sealed.

Although many different multilayer processes could be used to fabricate the reinforcing regions, the following steps can produce a suitable multilayer reinforcing region. Several layers of non-vulcanized polymer (e.g., non-vulcanized silicone) may be layered against the surface of expandable region 130. A reinforcing layer of a polyester mesh or other low compliance material may be layered among the backing layers. These multiple layers may be laid up in a variety of arrangements to achieve a desired level of reinforcement balanced against the layer thickness and the flexibility of the expandable body. With the layers laid up in the preferred arrangement, pressure and heat are applied to vulcanize the non-vulcanized materials. The layers bond to form non-separable layers. As an example, the layers are vulcanized in a vulcanizing press at about 175 degrees C. and about 100 pounds per square inch (psi) pressure for about twenty minutes, although these temperatures, pressures, and times may vary.

In some embodiments, reinforcing regions can be formed within the walls of expandable region 130. One method for forming reinforcing regions within the expandable region wall is to include one or more reinforcing layers during the fabrication of the expandable region wall. For example, during one type of dip-molding process for fabricating the expandable region, a mandrel or other form for making the body is repeatedly dipped into a polymeric solution to build up the thickness of the expandable region wall. During one or more of such dipping steps, a reinforcing layer, such as but not limited to a polyester mesh or other low compliance material, is positioned on the outer surface of the uncompleted expandable region. Subsequent dipping steps increase the thickness of the expandable region wall and cover the reinforcing layer or layers. At the conclusion of the dipping steps, the expandable region can be cured, whether by vulcanization or other curing process, to for the expandable region having a reinforcing region within the expandable region wall.

In some embodiments, reinforcing regions can be formed by varying the wall thickness of certain regions of the expandable region. For example, during one type of dip-molding process for fabricating the expandable region, a mandrel or other form for making the body is repeatedly dipped into a polymeric solution to build up the thickness of the expandable region wall. During the dipping process, certain regions of the uncompleted expandable region are subject to further dipping while other regions are not. The regions of the expandable region that have been subject to more dipping steps than other regions will have a greater wall thickness. Such areas of greater wall thickness form the reinforcing regions of the expandable region.

In certain embodiments, reinforcing regions are formed using some or all of the above methods or combinations thereof. The reinforcing regions of a single expandable region may be formed from one or more of the above methods, their equivalents, or combinations thereof.

Referring now to both FIGS. 2 and 2A, planar body 110 includes atraumatic region 140. Atraumatic region 140 is at or near the edges of device 100 and can extend around as much as the entire periphery of device 100 or less than the entire periphery. Atraumatic region 140 can be a series of discrete regions or a continuous region. In certain embodiments, atraumatic region 140 is characterized by: (i) a rounded, bulbous, or generally blunt shaped cross-section; (ii) a comparatively low hardness; (iii) a comparatively high flexibility; (iv) a lack of sharp or hard edges; (v) or combinations thereof.

According to embodiments of the invention, atraumatic region 140 is designed to reduce the likelihood of trauma to the tissue near device 100. Atraumatic region 140 can be fabricated according to any suitable method that provides the characteristics listed herein. Suitable methods for forming atraumatic region 140 include methods suitable for forming a bead of compliant material at or near the edge of planar body 110, including but not limited to bonding tubing to the edge of the device using vulcanization or other adhesion methods or liquid injection molding the bead area.

Referring again to FIGS. 2 and 2A, tongue region 120 and any tongues can optionally include expandable regions. Such expandable regions can be connected to expandable region 130 or can be separately connected to connector 190. Such expandable regions may apply constraining or constrictive forces about the posterior surface of the stomach.

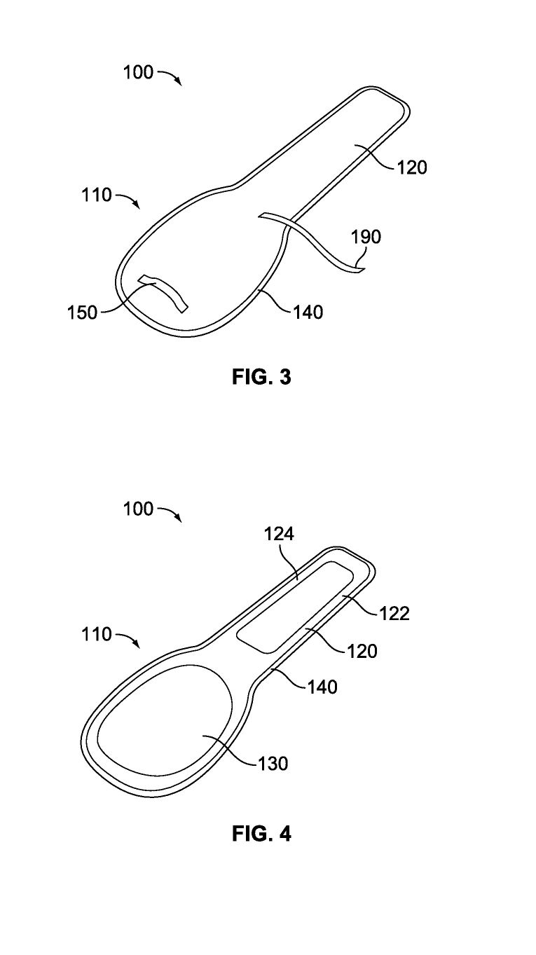

Referring now to FIG. 3, which illustrates a perspective view of the exterior side of device 100 where exterior refers to the side of device 100 not in contact with the surface of the stomach. The view in FIG. 3 illustrates additional features of certain embodiments of device 100. Planar body 110 includes fastener 150 on the exterior side of device 100. Fastener 150 is depicted here as a band under which at least a portion of tongue region 120 can pass. In general, fastener 150 is dimension and configured to reversibly couple with at least a portion of tongue region 120 in a substantially fixed manner. Fastener 150 and at least a portion of tongue region 120 can be fitted with complementary fixation parts, such as hook and loop, stud and socket, and other equivalent types of fixations devices. In certain embodiments, tongue region 120 can include wings, serrations, barbs, or similar fixation structures that allow tongue region 120 to pass though fastener 150 in one direction only. In any of the embodiments in which tongue region 120 includes fixation devices or structures, such fixation devices or structures may be arranged longitudinally relative to the long axis of tongue region 120 such that a selected amount of tongue region 120 may be passed though or otherwise fastened to fastener 150. The amount of tongue region 120 that is passed through or otherwise fastened to fastener 150 is one factor that controls the degree of constraining or constricting of the stomach by device 100. It is understood that while fastener 150 is illustrated in FIG. 150 as a band-type structure, fastener 150 can be configured in other functionally-equivalent ways. It is further understood that fastener 150 can be a region of planar body 110 which includes one or more fixation device or structures. For example, a region of planar body 110 could have an area of loop strips or patches designed to fasten with an complementary area of hook strips of patches on tongue region 120.

FIG. 4 illustrates a perspective view of device 100 according to certain embodiments. As illustrated in FIG. 4, tongue region 120 in configured to have tongue 122 and tongue 124. Tongue 122 and 124 converge at or near the point where tongue region 120 meets the area of planar body 110 which includes expandable region 130. Tongue 122 and 124 are depicted in FIG. 4 as also converging at the far end of tongue region 120, but it is understood that tongue 122 and 124 need not converge at the far end of tongue region 120. Further, FIG. 4 illustrates tongue region 120 have two tongues, but it is understood that tongue region 120 could have more, such as three, four, five, or six tongues. One potential advantage offered by embodiments having multiple tongues rather than a single large tongue is that multiple tongues may be easier to position about the posterior side of the stomach. Further, although FIG. 4 illustrates tongue region 120 and tongues 122 and 124 as having generally rectangular configurations, it is understood that these feature may have other configurations, such as but not limited to being tapered in width toward one end or being tapered in width toward the middle.

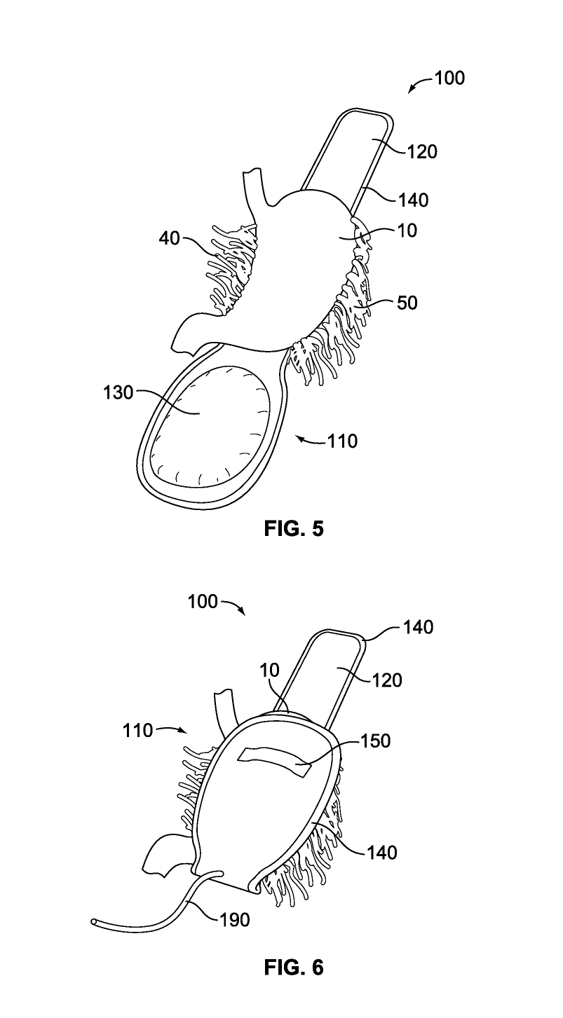

FIG. 5 illustrates a perspective view of device 100 being advanced into position around stomach 10 according to embodiments of the invention. FIG. 5 illustrates tongue region 120 being advanced upward about the posterior surface of stomach 10. Alternatively, device 100 can be designed so that the tongue region 120 is advanced around the inner curvature of the stomach 20. Prior to advancing device 100 around stomach 10, some of the connective tissue along outer curvature 50 is dissected, but the connective tissue and blood vessels near and along inner curvature 40 can be left intact. During the advancement of device 100 posterior to stomach 10, atraumatic regions 140 can help reduce or minimize tissue trauma. Alternatively, a guide-wire-like device or a guide tube (that may contain a flexible endoscope) with a blunt tip can used to establish the pathway behind or around the stomach such that the Implant can be advanced and guided in place via the guide-wire-like device.

FIG. 6 illustrates a perspective view of device 100 being positioned to constrain or constrict stomach 10 according to embodiments of the invention. FIG. 6 illustrates planar body 110 being folded about an inferior region of stomach 10 to come into contact with the anterior surface of stomach 10. In certain embodiments, device 100 is dimensioned and configured to contact substantially all of the anterior surface of stomach 10 and to maintain such contact when stomach 10 is full and when stomach 10 is empty. It is understood that in some embodiments device 100 can be dimensioned and configured to contact less than substantially all of the anterior surface of stomach 10 and still provide suitable constraining and constricting forces. Preferably, device 100 is dimensioned and configured to contact at least about half of the anterior surface area of the stomach.

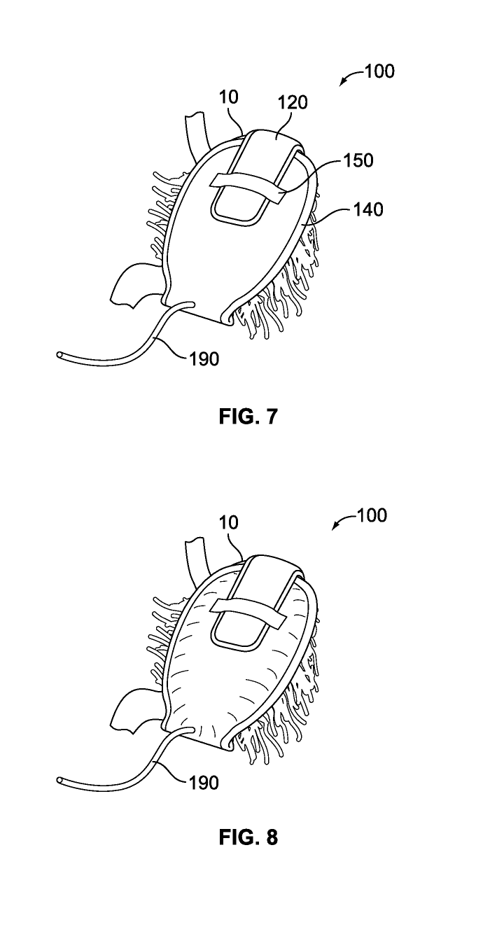

FIG. 7 illustrates a perspective view of device 100 being fastened into position to apply constraining or constricting forces to stomach 10 according to embodiments of the invention. FIG. 7 illustrates tongue region 120 as having been folded about a superior region of stomach 10. At least a portion of tongue region 120 passes through fastener 150 to hold device 100 in place in a substantially fixed manner. Tongue region 120 and fastener 150 are fastened together using any suitable fastening device or structures as described herein.

FIGS. 5, 6, and 7 do not depict the portion of tongue region 120 that is positioned about the posterior surface of stomach 10. Such portion of tongue region 120 can be any of the embodiments of the tongue region 120 disclosed herein. Specifically, the portion of tongue region 120 that is positioned about the posterior surface of stomach 10 can include multiple tongues. Preferably, in the case where tongue region 120 includes multiple tongues, the tongues converge at or near the place where tongue region 120 folds about an inferior region of stomach 10. It is also preferable that multiple tongues converge at or near the place where tongue region 120 folds about a superior region of stomach 10.

FIG. 8 illustrates a perspective view of device 100 being expanded to apply constraining or constricting forces to stomach 10 according to embodiments of the invention. Expandable region of device 100 is positioned in contact with at least a portion of the anterior surface of stomach 10 and expanded via connector 190 using the method described herein. Device 100 is expanded to an extent sufficient to constrain or constrict stomach 10 and can be adjusted subsequent to implantation.

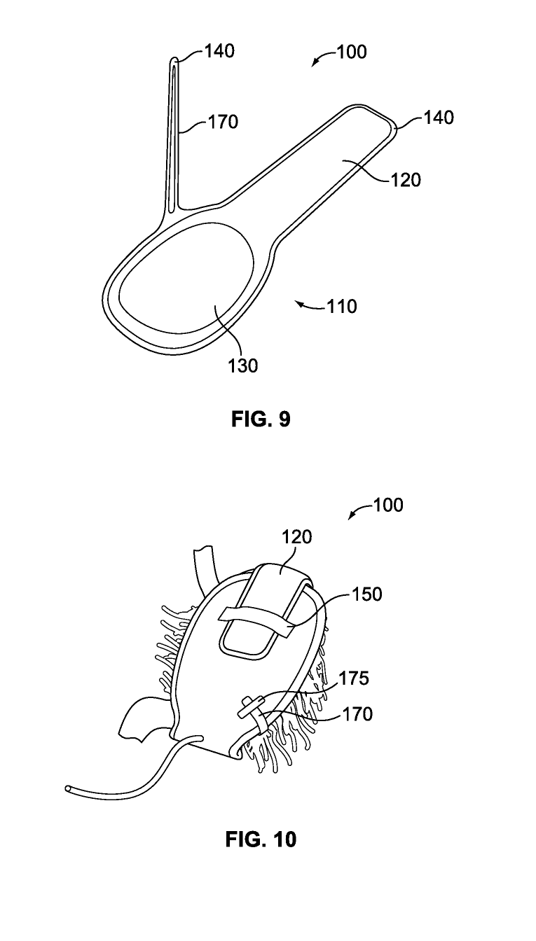

FIG. 9 illustrates a perspective view of device 100 having lateral retainer 170 according to embodiments of the invention. Lateral retainer 170 is connected to at least one edge of planar body 110. FIG. 9 depicts lateral retainer 170 connected to the edge of planar body 110 that will be placed at or near the inner curvature of stomach but it is understood that lateral retainer 170 may be connected instead to the opposite edge of planar body 110. Lateral retainer 170 is designed to be similar to tongues 122 and 124 of FIG. 4. For example, lateral retainer 170 may include one or more atraumatic regions 140.

FIG. 10 illustrates a perspective view of device 100 being fastened into position to apply constraining or constricting forces to stomach 10 according to embodiments of the invention. FIG. 10 illustrates tongue region 120 as having been folded about a superior region of stomach 10 and lateral retainer 170 as having been folded about a region of the inner curvature of stomach 10 and again folded about a region of outer curvature of stomach 10. At least a portion of tongue region 120 passes through fastener 150 to hold device 100 in place in a substantially fixed manner. Similarly, at least a portion of lateral retainer 170 passes through lateral retaining fastener 175 to further hold device 100 in place in a substantially fixed manner. Lateral retainer 170 and lateral retaining fastener 175 are fastened together using any suitable fastening device or structures as described herein. One advantage of lateral retainer 170 is that it may help maintain the position of device 100 about stomach 10 by providing a retention forces in a direction somewhat transverse to the retention forces provided by tongue region 120. It is understood that the relative positions of lateral retainer 170 and lateral retaining fastener 175 could be flipped such that lateral retainer 170 is attached to device 100 at the side of device near the outer curvature stomach 10. In such embodiments, lateral retainer 170 is first folded about a region of the outer curvature of stomach 10 and then about a region near the inner curvature of stomach 10 before being fastened to lateral retaining fastener 175. Further, it is understood that more than one lateral retainer 170 and lateral retaining fastener 175 may be attached to device 100 in certain embodiments and in such embodiments the multiple lateral retainers may be attached to the same side or different sides of device 100.

Tongue regions, tongues, and lateral retainers of embodiments of the invention are designed to be folded about certain areas of the stomach. Folding of these features can be accomplished through a folding region of the device. A folding region can be a area of the planar body which is comparatively thinner, comparatively more compliant, or otherwise adapted to be relatively easily folded about the stomach. Optionally, the folding region may be lacking the expandable region, thus achieving a thinner and more compliant section in the folding region. The folding region may optionally contain additional reinforcing regions. The folding region may optionally contain atraumatic regions or surfaces to prevent or reduce the incidence of pinching, binding, or otherwise inuring tissue in the area of the fold.

One of the novel aspects of certain embodiments of the invention is the configuration of the expandable region. The expandable region is dimensioned and configured to occupy a relatively large surface area of the stomach without occupying a commensurately large volume of space in the abdominal cavity. For example, as compared to an expandable device of the prior art having a generally spherical cross-section, the expandable region described herein occupies less abdominal volume while occupying a similar or greater surface area of the stomach. As compared to prior art expandable devices, expandable region of certain embodiments of the invention can be said to have a low profile. The expandable region is included in the generally planar shape of the device. Expandable regions of certain embodiments have a higher ratio of stomach-contacting surface area to internal volume than certain prior art expandable devices. The low profile of the expanded device and relatively high surface area to internal volume ratios can facilitate the distribution of constraining or constricting forces about the stomach while reducing the compression of, dissection or, or other trauma to the surrounding blood vessels and connective tissue.

In some embodiments in which multiple expandable regions are present in a single planar body, such expandable regions of a single planar body may have the same surface area to internal volume ratio, the expandable regions may have different ratios, or some of the expandable regions may have the same ratio and some may have different ratios. In embodiments, the planar body has expandable regions of varying shapes and sizes and the surface area to volume ratios of each such expandable region may be the same or different. That is, an expandable region with a given shape may have the same surface area to volume ratio as a expandable region with a different shape. Or, the ratio of differently-shaped expandable regions may be different.

For the expandable regions, the surface area of the expandable region that is in contact with the stomach can range from about 24 cm² (9 square inches) to about 345 cm² (53 square inches). Preferably, the surface area of the expandable region that is in contact with the stomach can range from about 86 cm² (13 square inches) to about 195 cm² (30 square inches). The internal volume of the expandable region can range from about 38 cc (2.4 cubic inches) to about 686 cc (42 cubic inches). Preferably, the internal volume of the expandable region can range from about 89 cc (5.4 square inches) to about 310 cc (19 square inches). In certain embodiments, the ratio of the surface area of the expandable region that is in contact with the stomach to the internal volume of the expandable region can range from about 9:1 to about 0.6:1. Preferably, ratio of the surface area to the internal volume of the expandable region can range from about 5:1 to about 1.1:1.

The expandable region of the planar body of devices of certain embodiments can be designed to selectively constrain or constrict some area of the stomach more than others. Such selective distribution of forces can be accomplished using multiple expandable regions in a single planar body, by varying the compliance of the reinforcing region or regions, by combinations of these methods, or by equivalent methods. Device of certain embodiments can apply comparatively more force, compression, or constriction about the fundus region of the stomach. For example, an area of expandable region placed near the fundus may preferably be designed to jut into or expand into the stomach wall in that region such that the volume of the stomach near the fundus is decreased comparatively more than other regions of the stomach. Similarly, an area of expandable region placed near the fundus may exert comparatively greater force on the fundus region of the stomach by jutting into, expanding into, or otherwise distending the stomach wall in the fundus region.

The device of embodiments of the invention preferably moves with the stomach even though it is not physically anchored to the stomach by staples, sutures, or other conventional means. Of course, it is understood that such convention means of anchoring are within the scope of the invention. The folding of the tongue region about the inferior and superior regions of the stomach and the folding of one or more lateral retainers about the inner and outer curvature of the stomach in certain embodiments of the device facilitate fitting the device about the stomach regardless of the presence of food in the stomach. The expandability of the expandable region of the device and the presence of reinforcing regions of the device are also designed to maintain contact of the device with the stomach.

Although the devices of certain embodiments of the invention are effectively anchored to the stomach by virtue of their dimensions and configuration, it is understood that conventional physical anchors that bond with or otherwise physically interact with the stomach or the abdominal wall may optionally be used to further anchor the device to the stomach or the abdominal wall, in particular the anterior abdominal wall. For example, tissue ingrowth meshes can be included on or near a surface of the device. An ingrowth layer (preferably, but not necessarily made of a layer of mesh or velour, such as a polyester fiber configured and dimensioned to encourage tissue growth into it) is laid on the outside surface of the expandable body. Multiple ingrowth layers can be used, each having a relatively less dense or more dense weave than the first ingrowth layer. The layers can be bonded to the expandable body using the multilayer vulcanization techniques discussed herein or other equivalent bonding techniques, including adhesive bonding and suturing. The tissue ingrowth mesh can be sutured to the abdominal wall (and/or optionally to the stomach) to anchor the device. Tissue adhesives such as fibrin glue can be used to help fixate the Implant to the stomach or abdominal wall to prevent relative motion and help with the ingrowth process.

One advantage of embodiments of the inventions is that these devices can provide significant coverage of the surface area of the stomach after they have been advanced and positioned about the stomach. However, as compared to other device designs, devices dimensioned and configured according to embodiments of the invention can be advanced and positioned while causing comparatively less trauma to the blood vessels and tissue on or near the stomach. The low profile and high surface area to volume ratio of the planar body of certain devices can also enable such devices to provide the compressive and constrictive forces while causing comparatively less trauma than prior art devices.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A device for the treatment of obesity, the device comprising:
a planar body having a configuration suitable for being positioned about the exterior of the stomach of a patient, the body comprising an expandable region and a tongue region.

2. The device of claim 1 wherein the body further comprises a fastening region configured to fasten the tongue region.

3. The device of claim 1 wherein the body further comprises a folding region positioned between the expandable region and the tongue region.

4. The device of claim 1 wherein the expandable region further comprises a reinforcing region.

5. The device of claim 1 wherein the expandable region is configured to contact at least of the anterior surface area of the stomach.

6. The device of claim 1 wherein the expandable region is configured to contact substantially all of the anterior surface area of the stomach.

7. The device according to claim 1 wherein the body maintains a substantially planar configuration following expansion of the expandable region.

8. The device according to claim 1 wherein the body further comprises atraumatic edges.

9. The device according to claim 1 wherein the planar body further comprises a lateral retainer.

10. A method for the treatment of obesity, the method comprising:
accessing an extra-gastric abdominal space of a patient;
introducing a device into the extra-gastric abdominal space, the device comprising
an expandable region having a substantially planar configuration suitable for being positioned about the exterior of the stomach of the patient; and
a tongue region;
positioning the expandable region to be in contact with at least a portion of a first surface of the stomach;
positioning the tongue region to be in contact with at least a portion of a second surface of the stomach, wherein one of the first surface and the second surface is an anterior surface of the stomach and the other is a posterior surface of the stomach;
fastening the tongue region to the expanding region; and
expanding the expandable region.

11. The method of claim 10 further comprising positioning the device about the stomach wherein upon expansion the device preferentially constrains the fundus.

12. The method of claim 10 further comprising positioning the device about the stomach wherein upon expansion the device preferentially constrains an inferior region of the stomach.

13. The method of claim 10 wherein the device further comprises a folding region positioned between the expandable region and the tongue region.

14. The method of claim 13 further comprising positioning the folding region near an inferior region of the stomach.

15. The method of claim 10 further comprising folding the tongue about a superior region of the stomach.

16. The method of claim 10 wherein the device further comprises a lateral retainer.

17. The method of claim 16 further comprising folding a lateral retainer about the inner curvature of the stomach.

18. A device for the treatment of obesity, the device comprising:
a planar body having a configuration suitable for being positioned about the exterior of the stomach of a patient, the body comprising at least one expandable region having surface area to internal volume ratio of greater than about 1.1:1 when expanded.

19. The device of claim 18 wherein the body further comprises a tongue region.

20. The device of claim 18 wherein the body further comprises a lateral retainer.
